(19) United States
(12) Patent Application Publication
(10) Pub. No.: US 2010/0285485 A1
(43) Pub. Date: Nov. 11, 2010

(54) PRIMER, PROBE, DNA CHIP CONTAINING THE SAME AND METHOD FOR DETECTING HUMAN PAPILLOMAVIRUS AND DETECTION KIT THEREOF

(76) Inventors: Woon-Won Jung, Gyeonggi-go (KR); Hyun-Sook Kim, Seoul (KR)

Correspondence Address: MERCHANT & GOULD PC, P.O. BOX 2903, MINNEAPOLIS, MN, 55402-0903, US

(21) Appl. No.: 12/811,280

(22) PCT Filed: Dec. 31, 2008
(86) PCT No.: PCT/KR08/07836

§ 371 Date: Jun. 30, 2010

(30) Foreign Application Priority Data
Dec. 31, 2007 (KR) 10-2007-0141257
Dec. 30, 2008 (KR) 10-2008-0137163

(51) Int. Cl. C12Q 1/68; C07H 21/04

(52) U.S. Cl. 435/6; 536/24.33; 536/24.32

(57) ABSTRACT

The present invention discloses a primer, a probe, a DNA chip, and a test kit for diagnosis and genotyping of Human Papilloma Virus (HPV) as well as a method for testing HPV genotype. According to the present invention, it is possible to screen HPV genotypes with high sensitivity and specificity, and to diagnose infection by multiple HPV types which was not possible in other conventional HPV testing methods

### TECHNICAL FIELD

The present invention relates to a primer, a probe and DNA chip containing them for genotyping of Human Papillomavirus (HPV). Specifically, the present invention relates to a PCR primer for amplification of HPV genes, a probe which specifically binds with the amplified genes, and a testing kit and method using them for diagnosis and/or test of HPV genes.

### BACKGROUND ART

HPV has been found in papilloma of cottontail rabbit, and known as one of oncoviruses, a DNA virus on a double-helix with a size of about 45-55 nm and 8 kb. HPV belongs to Papovavirus family, and it is generally found in various kinds of animals other than rabbit, causing infections in the form of warts or condyloma in human.

HPV has been known to cause cervical cancer in women, and be closely related to other malignant tumors such as skin cancer, laryngeal cancer or the like. HPV infection is very commonly occurred in women, approximately 75% of women get infected as they become sexually active. Cervical cancer can be developed 10-20 years after a certain HPV infection. 20˜25% of infections may progress to a precancerous stage.

At present, approximately 120 types of different HPV genotypes have been known. Among HPV types, types prevalently infecting the genital area are following 45 types: HPV-16, -31, -33, -35, -52, -58, -67, -40, -43, -7, -32, -42, -6, -11, -74, -44, -55, -13, -61, -72, -62, -2, -27, -57, -3, -28, -29, -10, -54, -18, -39, -45, -59, -68, -70, -26, -69, -51, -30, -53, -56, -66, -34, -64 and -73. Among them, HPV types being closely associated with cervical cancer are: as a high-risk group of 22 types, HPV-16, -18, -26, -30, -31, -33, -35, -45, -51, -52, -53, -56, -57, -58, -59, -61, -67, -68, -70, -73 and -74 and as a low-risk group, HPV-2a, -3, -6, -10, -11, -32, -34, -40, -43, -44, -54, -55, -66 and -69. 90% HPV types belonging to the high risk group are detected in epithelial tissues of cervical tumors.

Knowing the HPV types infecting a patient, it is possible to expect how the infection would progress to diseases in the future. Therefore, it is important to test the presence of HPV and confirm the exact types thereof.

As conventional methods for diagnosis of HPV infection, there are Southern blot hybridization, dot (slot) blot hybridization (ViraPap), in-situ hybridization, hybrid capture system, PCR (polymerase chain reaction) and the like.

The conventional diagnosis methods are advantageous in terms of being capable of partial quantification and convenience. However, for a diagnosis purpose, there are some disadvantages such that they require long time to get the result and complex experimental techniques, and diagnosis of HPV genotypes is not so convenient.

A DNA chip method, which has been recently developed, is a method combined with DNA amplification, being capable of testing the HPV genotypes from a large amount of samples in convenient way, without problems of production economy or process time. Such advantages make this method very useful in early diagnosis, prevention and prognosis of cervical cancer.

Since 2003, US FDA has recommended both PAP smear testing and HPV infection test at the same time for testing cervix cancer. At present, vaccines for HPV-16 and HPV-18 have been developed and now available. For monitoring effects thereof, a device (kit) or a method for diagnosing HPV genotype is needed. Further, since tests on infection by various HPV genotypes are essential, a HPV diagnosis device (kit) and a test method for determining HPV genotypes are further required, at present.

### DISCLOSURE

### Technical Solution

The present invention is to provide a primer used in PCR for HPV amplification.

Further, the present invention is to provide a probe for testing HPV genotypes with enhanced specificity.

Still further, the present invention is to provide a DNA chip and kit for testing several tens of HPV genotypes in rapid way.

Further, the present invention is to provide a method for testing several tens of HPV genotypes at once through a single test.

### ADVANTAGEOUS EFFECTS

According to the present invention, it is possible to identify 36 HPV types present in cervical cells with enhanced specificity and further to detect multiple infections by different HPV types. The present invention can test HPV genotypes in a reduced time, taking about 6 hours or less from DNA extraction from cervical cell specimen to identification of the HPV genotype by using a DNA chip.

### DESCRIPTION OF DRAWINGS

FIG. 1 is a view showing a DNA chip for a HPV genotype test according to one embodiment of the present invention.

FIG. 2 is a view showing the gene amplification result by using a primer according to the present invention, JK1 set. HPV genotypes are as follows: from left to right, 6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84, 90, positive control, and negative control.

FIG. 3 is a view showing the gene amplification result by using a primer according to the present invention, JK2 set. HPV genotypes are as follows: from left to right, 6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84, 90, positive control, and negative control.

FIG. 4 is a view showing the gene amplification result by using a control primer set. HPV genotypes are as follows: from left to right, 6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84, 90, positive control, and negative control.

FIGS. 5 and 6 are the results of HPV genotype tests with a DNA chip according to the present invention, appeared by a chip scanner.

FIG. 7 is a DNA extraction reagent kit according to one embodiment of the present invention.

FIG. 8 is a reagent kit containing a PCR primer according to one embodiment of the present invention.

FIG. 9 is a DNA chip kit for testing HPV genotype according to one embodiment of the present invention.

FIGS. 10 to 22 are views showing the base sequencing results of PCR products according to each HPV genotype, obtained from Example 1-1.

### MODE FOR INVENTION

In one aspect, the present invention provides a primer for HPV (Human Papilloma Virus) amplification, which contains at least one base sequence selected from the group consisting of SEQ.ID.NO:1 to SEQ.ID.NO:6.

The base sequence may have a fluorescent dye attached to 5′ end thereof. The fluorescent dye can be selected from the group consisting of Cy3, Cy5, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), x-rhodamine, fluorescein isocyanate (FITC) and Texas red.

In another aspect, the present invention provides a probe which forms complementary binding with HPV, containing at least one oligonucleotide selected from the group consisting of oligonucleotide having base sequences represented by SEQ.ID.NO:9 to SEQ.ID.NO:44, and oligonucleotide having base sequences complementary to said sequences.

The oligonucleotide may correspond to the following HPV types: HPV-6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84 and 90.

To the 5′ end of the base sequences represented in SEQ.ID.NO:9 to SEQ.ID.NO:44, an amine group can be attached.

In still other aspect, the present invention provides a DNA chip for testing HPV genotypes, containing the above-described probe.

In another aspect, the present invention provides a kit for detecting or testing HPV genotype, containing:

a reagent for DNA extraction;

a PCR primer for amplification of the extracted DNA; and

a DNA chip for testing HPV genotype containing a probe according to any one of claims 4 to 6.

The primer for PCR can contain at least one base sequence selected from the group consisting of SEQ.ID.NO:1 to SEQ.ID.NO:6.

The primer for PCR can be a set selected from: a set consisting of SEQ.ID.NO:1 and SEQ.ID.NO:2 (referred to ‘JK1 set’); a set consisting of SEQ.ID.NO:3 and SEQ.ID.NO:4 (referred to ‘JK2 set); and a set consisting of SEQ.ID.NO:5 and SEQ.ID.NO:6 (referred to ‘JK3’).

In still other aspect, the present invention provides a method for testing HPV genotype, comprising the steps of:

extracting DNA from cells taken from the cervix;

amplifying the DNA by PCR involving a primer for HPV amplification;

hybridization of the amplified DNA to a DNA chip containing a HPV probe which contains at least one oligonucleotide selected from the group consisting of oligonucleotide having base sequences represented by SEQ.ID.NO:9 to SEQ.ID.NO:44 and oligonucleotide having base sequences complementary to said sequences; and

determining the HPV genotype through the product specifically hybridized with the probe.

The method can further include, in the PCR amplification, a beta-globin primer set represented by SEQ.ID.NO:7 and SEQ.ID.NO:8, as a control.

In the hybridization of the amplified DNA, an amine group can be attached to the 5′ end of the base sequences represented in SEQ.ID.NO:9 to SEQ.ID.NO:44.

In the step of determining the HPV genotype, the HPV genotype can be selected from the group consisting of HPV-6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84, 90 and mixtures thereof.

The present invention is comprised of the following steps: amplification of DNA by PCR involving a primer for HPV amplification; hybridization of the amplified DNA to a DNA chip containing a HPV probe; and determining HPV genotype, by assaying the product specifically hybridized with the probe. For achieving the invention, the present invention provides a novel primer, a probe, a DNA chip and a test kit. Hereinafter, the present invention is further illustrated in detail.

The primer contains at least one base sequence selected from the group consisting of SEQ.ID.NO:1 to SEQ.ID.NO:6. The primer is 5′ primer or 3′ primer specific to HPV genes, as represented in Table 1 below.

TABLE 1

| SEQ. | | | |
|---|---|---|---|
| ID. No. | Primers | | Sequence (5′ → 3′) |
| 1 | JK1 | 5′ | 5′-AGT GGS TCT ATG GTW WCY TC-3′ |
| 2 | | 3′ | 5′-CAT AGT ATG TAW RTA KGY CAT-3′ |
| 3 | JK2 | 5′ | 5′-GCC ACA AYA ATG GYA TWT GYT-3′ |
| 4 | | 3′ | 5′-TGT AAA TCA TAT TCC TCM MCA |
| | | | TG-3′ |
| 5 | JK3 | 5′ | 5′-CCA CCT ATA GGG GAA CAC TGG-3′ |
| 6 | | 3′ | 5′-GAG GTA ACC ATA GAA CCA CT-3′ |

The base sequence can be easily detected, owing to a fluorescent dye being attached to 5′ end thereof. The fluorescent dye may be selected from the group consisting of Cy3, Cy5, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), x-rhodamine, fluorescein isocyanate (FITC) and Texas red, without being limited to these.

In the present invention, the primer can be one or more selected from the group consisting of SEQ.ID.NO:1 to SEQ.ID.NO:6. Preferably, the primer is used as a primer set selected from: JK1 set consisting of SEQ.ID.NO:1 and SEQ.ID.NO:2: JK2 set consisting of SEQ.ID.NO:3 and SEQ.ID.NO:4; and JK3 set consisting of SEQ.ID.NO:5 and SEQ.ID.NO:6.

In addition to the primer, the present invention may further include a beta-globin primer set represented by SEQ.ID.NO:7 and SEQ.ID.NO:8, as a control gene. To the end of the 5′ primer of beta-globin, a fluorescent dye can be attached.

The probe of the present invention contains at least one oligonucleotide selected from the group consisting of oligonucleotide having base sequences represented as SEQ.ID.NO:9 to SEQ.ID.NO:44, and oligonucleotide having base sequences complementary to said sequences, as shown in Table 2. As seen from the following Table 2, the probes of the present invention can specifically bind with 36 types of HPV genotypes in total.

To the 5′ end of the base sequences represented in SEQ.ID.NO:9 to SEQ.ID.NO:44, an amine group can be attached so as to be used in DNA chip fabrication. Such technique is well known in this field of art to which the present invention pertains, thereby eliminating detailed description thereof in this specification.

The expression ‘specifically hybridized’ used herein, is generally used in this field of art, referring to a reaction of forming complementary binding between bases. Such specific binding reaction is called ‘hybridization’.

The primer, as described in the above, amplifies HPV gene (DNA), and then thus amplified gene is specifically hybridized with the probe, resulting a product by which HPV genotype can be determined.

TABLE 2

| | HPV types with which | |
|---|---|---|
| SEQ. | the probe is | |
| ID. | specifically | |
| No. | hybridized | Sequence (5′-3′) |
| 9 | HPV6 | ACC AAT TCT GAT TAT AAA GAG TAC ATG CGT |
| 10 | HPV11 | TTC AGA TTA TAA GGA ATA CAT GCG CCA TGT |
| 11 | HPV16 | CTA CAT ATA AAA ATA CTA ACT TTA AGG AGT |
| 12 | HPV18 | CTC CTG TAC CTG GGC AAT ATG ATG CTA CCA |
| 13 | HPV31 | CTA CAT TTA AAA GTA GTA ATT TTA AAG AGT |
| 14 | HPV32 | GTG CTA CTG TAA CAA CTG AAG ACA CAT ACA |
| 15 | HPV33 | CAC ACA AGT AAC TAG TGA CAG TAC ATA TAA |
| 16 | HPV34 | TGC AAA CAG TAA TTT TAA GGA ATA CCT CAG |
| 17 | HPV35 | CTG CTG TGT CTT CTA GTG ACA GTA CAT ATA |
| 18 | HPV39 | TGA TCC TTC TAA GTT TAA GGA ATA TAC CAG |
| 19 | HPV40 | TAA TAA CAG TAA TTT CAA GGA ATA TTT GCG |
| 20 | HPV42 | CAG CTG CTA ATT TTA AGG AAT ATT TAA GAC |
| 21 | HPV43 | TAT GAC AAT GCA AAG TTT AAG GAA TAC TTG |
| 22 | HPV44 | TAC TAG TGA ACA ATA TAA GCA ATA CAT GCG |
| 23 | HPV45 | CCT ACT AAG TTT AAG CAG TAT AGT AGA CAT |
| 24 | HPV51 | TCC AAG TAA CTT TAA GCA ATA TAT TAG GCA |
| 25 | HPV52 | TGA AAA TTT TAA GGA ATA CCT TCG TCA TGG |
| 26 | HPV53 | AGC AAA TTA AAC AGT ATG TTA GAC ATG CAG |
| 27 | HPV54 | TTC TGA CTT TAG GGA GTA TAT TAG ACA TGT |
| 28 | HPV56 | TGC ACG AAA AAT TAA TCA GTA CCT TAG ACA |
| 29 | HPV58 | AAA AAT GAT AAT TTT AAG GAA TAT GTA CGT |
| 30 | HPV59 | CAC ACC TAC CAG TTT TAA AGA ATA TGC CAG |
| 31 | HPV61 | TGT ATC TGA ATA TAA AGC CAC AAG CTT TAG |
| 32 | HPV62 | TCC ACT GCT GCA GCA GAA TAC AAG GCT ACC |
| 33 | HPV66 | ATG ATG CCC GTG AAA TCA ATC AAT ACC TTC |
| 34 | HPV67 | TGT TCT GAG GAA AAA TCA GAG GCT ACA TAC |
| 35 | HPV68 | TTA TGA TCC TAA TAA ATT TAA GGA ATA TAT |
| 36 | HPV69 | TAA ACC ATC AGA TTA TAA GCA GTT TAT AAG |
| 37 | HPV70 | TAG CCC TAC AAA GTT TAA GGA ATA TAC TAG |
| 38 | HPV71 | GCC TCT AGT TTC ATG GAA TAT TTG AGA CAT |
| 39 | HPV72 | GCG TCC TCT GTA TCA GAA TAT ACA GCT TCT |
| 40 | HPV81 | GGC CTC TAA CTT TAA GGA ATT TCT GCG CCA |
| 41 | HPV82 | CCA TCT GTT GCA CAA ACA TTT ACT CCA GC |
| 42 | HPV83 | CAC AGG CTA ATG AAT ACA CAG CCT CTA ACT |
| 43 | HPV84 | CAC CGA ATC AGA ATA TAA ACC TAC CAA TTT |
| 44 | HPV90 | GAC ACA TAC AAG GCT TCC AAT TTT AAA GAG |

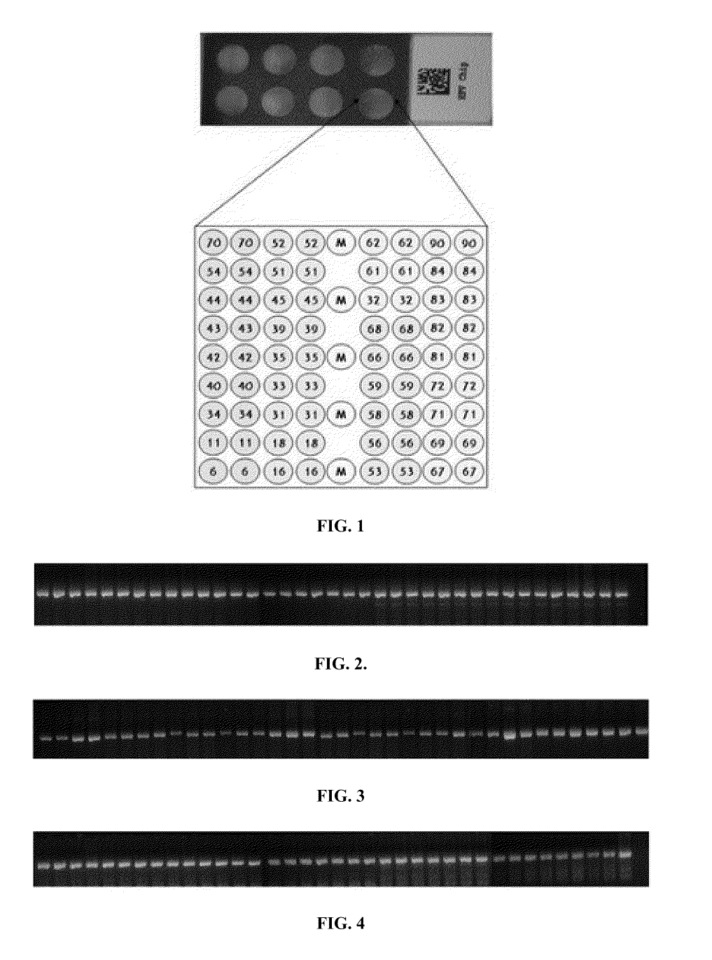

According to the present invention, a DNA chip for a genotype test may be fabricated by using said probe (FIG. 1). It will be further illustrated through the following examples.

In another aspect, the present invention provides a kit for detecting or testing HPV genotype, containing: a reagent for DNA extraction; a PCR primer for amplification of the extracted DNA; and a DNA chip for testing HPV genotype, containing the above-described probe.

Further, the present invention provides a method for testing HPV genotype, including the steps of: extracting DNA from cells taken from the cervix; amplifying the DNA by PCR involving a primer for HPV amplification; hybridization of the amplified DNA to a DNA chip containing the above-described HPV probe; and determining the HPV genotype through the product specifically hybridized with the probe.

Specifically, a method for detecting HPV genes and/or testing HPV genotypes according to one embodiment of the present invention may include the following procedure, however it is not just limited to this example.

1) centrifuging the cervix cells taken from a vaginal swab to collect cells and lysing cells with a solution for cell lysis so as to obtain DNA,

2) amplifying a portion of the resulted DNA by using the 5′ primer and 3′ primer of the present invention, which are the primers specific to HPV genes, for HPV gene amplification, and

3) applying the amplified HPV genes to a 2.5% agarose gel, and determining the PCR products by the presence of fluorescence visualized by Et Br.

A method for testing HPV genotypes by using a DNA chip according to one embodiment of the present invention may include the following procedure, however it is not limited to this example.

1) centrifuging cervix cells taken from a vaginal swab to collect cells and lysing cells with a solution for cell lysis so as to obtain DNA,

2) amplifying a portion of the resulted DNA by using the 5′ primer and 3′ primer of the present invention, which are primers specific to HPV genes, for HPV gene amplification,

3) fabricating a DNA chip for testing genotypes of the amplified HPV genes, by synthesizing oligonucleotides of 36 types of HPV probes which specifically bind to the 36 types of HPV genes, to the size as much as about 30 nucleotides,

4) placing each probe on a glass panel for chip fabrication in array at a certain interval by using a Microarrayer, and washing and fixing them to fabricate a chip,

5) hybridizing the PCR products of HPV genes from 2) to the resulted chip at a certain temperature and washing it so as to remove those unspecifically bound therefrom,

6) determining the HPV genotypes specifically bound to the HPV probes by using a chip scanner.

Further, a kit for testing HPV genotypes according to one embodiment of the present invention may include a reagent, a device and equipment as follows, however it is not just limited to this example.

1) DNA extraction from cells:

\textcircled{1} buffer for cell lysis (7% chelex, 20 mM Tris HCl, 100 ug/ml proteinase K, 10 mM CaCl₂, pH 8.0)

2) PCR of HPV genes:

\textcircled{1} 20 pmol/μl of each primer set

\textcircled{2} 10×PCR buffer solution

\textcircled{3} Taq. DNA polymerase 250 unit (Solgent, South Korea)

\textcircled{4} 2.5 mM dNTP (dATP, dGTP, dCTP, dUTP)

\textcircled{5} Uracil-N-Glycosylase 1 U/μl

3) fabrication of a DNA chip for testing HPV genotypes:

\textcircled{1} 36 types of oligonucleotide probes of HPV genes, wherein an amine group is attached to 5′ end thereof (concentration: 50 pmol/μl)

\textcircled{2} a glass slide (silanated, amine-coated)

\textcircled{3} a 8-well hybridization chamber (Grace, US)

\textcircled{4} a blocking buffer (5×SSC, 1% BSA, 0.1% SDS)

\textcircled{5} isopropyl alcohol

\textcircled{6} distilled water

4) hybridization of PCR products of HPV genes with the HPV DNA chip:

\textcircled{1} a 8-sectioned DNA chip for testing HPV genotype, fabricated in the above 3)

\textcircled{2} hybridization buffer (0.4M Mes, 20 mM MgCl₂, 0.15% SDS, 0.15% BSA, pH 6.5)

5) Washing DNA chip completed with the hybridization:

\textcircled{1} washing solution I (2×SSC, 0.2% SDS)

\textcircled{2} washing solution II (0.1×SSC)

6) HPVDNA chip reading:

\textcircled{1} microarrayer scanner (GSI Lumonics, USA)

By fabricating a DNA chip for a HPV genotype test from the above-described reagents, devices and equipment, it is possible to determine HPV genotype of samples taken from the cervix. Further, based on such DNA chip, a kit for HPV gene detection or a HPV genotype test can be formed by the following construction. However, the following description is only one embodiment of the present invention, and the present invention is by no means limited by such examples.

1) A kit for extracting DNA from cells:

\textcircled{1} a 30 ml volume tube containing a gene extract solution (7% (w/v) chelex, 20 mM Tris HCl, 100 ug/ml proteinase K, 10 mM CaCl₂, pH 8.0)

\textcircled{2} a 30 ml volume tube containing a 10× washing solution (200 mM Tris-HCl, 50 mM KCl, 50 mM NaH₂PO₄, 50 mM Na₂HPO₄, pH 7.4)

2) A kit for PCR of HPV genes:

\textcircled{1} a 1.5 ml volume tube containing a primer set selected among JK 1, 2 and 3, at the concentration of 20 pmol/μl

\textcircled{2} a 1.5 ml volume tube containing 10×PCR buffer solution

\textcircled{3} a 1.5 ml volume tube containing Taq DNA polymerase 250 units (Tag., Solgent, South Korea)

3) A kit for a HPV genotype test of PCR products of HPV genes:

\textcircled{1} a DNA chip for a HPV genotype test

\textcircled{2} a multiwell hybridization chamber (8-well) (Sigma, US)

For diagnosis of HPV infection, kits 1) and 2) may be used, and for a HPV genotype test, kits 1), 2) and 3) may be used. According to the present invention, it takes about 6 hours from extraction of DNA from the cervix cells and HPV gene amplification by PCR to HPV genotype assay by using the HPV DNA chip. With one HPV DNA chip, 8 samples can be tested simultaneously.

Hereinafter, the present invention is further illustrated by the examples provided below. However, it should be understood that these examples only have illustrative purpose, by no means limiting the scope of the present invention.

### EXAMPLES

### Example 1

### Detection of HPV Genes by Using HPV Gene Amplification

1-1: DNA Extraction from Cervix Cell

A cytobrush sample preserved in 5 ml of a washing solution (PBS) was severely vortexed to separate the cervix cells therefrom. The cells were centrifuged at 1,300 g for 10 minutes, and the supernatant was removed therefrom. The cells were re-suspended in a washing buffer (200 mM NaCl, 50 mM KCl, 50 mM NaH2PO4, 50 mM Na2HPO4, pH 7.4), transferred to a 1.5 ml-volume microtube, and centrifuged at 1,300 g for 5 minutes. The supernatant was removed, and 200 μl of a chelex buffer (7% chelex, 20 mM Tris HCl, 100 μg/ml proteinase K, 10 mM CaCl₂, pH 8.0) were added thereto for re-suspension. The suspension was heated in a water bath at 55° C. for 3 hours, and then further heated at 95° C. for 20 minutes so as to eliminate the activity of proteinase K, finally obtaining DNA from the cervix cell.

1-2: HPV Gene Amplification by PCR

For amplifying the DNA obtained from the above 1-1, JK1 and JK2 primer sets as shown in Table 1 were used independently. As a control, a β-globin primer represented in SEQ.ID.NO: 7 and SEQ.ID.NO: 8 was used, wherein Cy5 fluorescent dye was attached to the 5′ end of the 3′ primer (5′-Cy5-CAA GAC AGG TTT AAG GAG ACC A-3′/5′-GGT TGG CCA ATC TAC TCC CAG G-3′).

For gene amplification (PCR), into a 0.2 ml PCR tube, 2.5 μl 10× buffer (750 mM Tris-HCl (pH9.0), 150 mM (NH₄)₂SO₄, 25 mM MgCl₂, 1 mg/ml BSA) 5 μl, 2.5 mM dNTP 2.0 μl, Taq polymerase (5 units) 0.3 μl, 20 pmolor JK2 primer set 1.0 μl, respectively, template DNA 5 μl were added, and by further adding distilled water thereto, 25 μl of total volume was made. For pre-denaturation, the amplified gene mixture was heat-treated at 50° C. for 3 minutes and then 95° C. for 15 minutes. PCR thereof was conducted at 95° C. for 60 seconds, 53° C. for 60 seconds, and 72° C. for 60 seconds, and this cycle was repeated 40 times. Finally, it was treated at 72° C. for 30 minutes (9700, Applied Biosystems).

The amplification of β-globin genes that was used as a control, was conducted under the conditions as same as above, except the different primer. After completing the PCR amplification, 3.0 μl of the amplified gene products was applied to a 2.5% agarose gel for electrophoresis, and the image analysis was carried out (imageanalyzer, alphascan). The time elapsed was one and half hour or so. The gene amplification results using JK1, JK2 and control as a primer, were shown in FIGS. 2 to 4. As seen from the results illustrated in FIGS. 2 to 4, the primer according to the present invention exhibited superior gene amplification effect.

### Example 2

### Fabrication of a DNA Chip for Diagnosis and Genotyping of HPV Genes

2-1: Fabrication of a DNA Chip for Testing HPV Genotype

By using Microarrayer (Bio-Robotics, UK), a silanated, amine-coated glass slide was divided into 8 sections, and 36 types of HPV genotype probes at the concentration of 50 pmol/μl, respectively, (base sequences were shown in Table 2) attached to each section in identical way.

The glass slide attached with the probes was kept in a dark place for 2 days; then baked at 80° C. for 4 hours or more; and subjected to shaking incubation in a blocking buffer (5×SSC, 1% BSA, 0.1% SDS) at 42° C. for 45 minutes. Isopropanol was added thereto and the mixture was subjected to shaking incubation for 1 minute. After removing the supernatant (reacted solution), shaking incubation in distilled water was carried out for 1 minute and it was repeated 5 times. The resulted slide was dried and kept in a closed container before use.

2-2: Testing Genotypes of PCT Products of HPV Genes by Using a DNA Chip for a HPV Genotype Test

1) Preparation of a DNA Chip for a HPV Genotype Test

To the HPV DNA chip fabricated in the above 2-1, a 8-well hybridization chamber (Grace, US) was rigidly fixed to obtain a 8-sectioned DNA chip (see, FIG. 1)

2) Hybridization of PCR Products of HPV Genes with the DNA Chip

For hybridization with the oligonucleotides spotted on the HPV DNA chip, PCR product 8 μl and 2× hybridization buffer 32 μl were added into a 1.5 ml tube, wherein the PCR product was denatured at 95° C. for 5 minutes, and the hybridization buffer 32 μl was allowed for reaction at 48° C. for 10 minutes just right before use. The hybridization buffer and the PCR product were well mixed together, and carefully added to each opening of a well on the DNA chip, (1 sample per well). The opening of the well was covered with Cellotape for preventing the hybridization mixture from being dried out, allowing hybridization to occur at 48° C. for 1 hour.

3) Washing the DNA Chip after Completion of Hybridization

The DNA chip completed with hybridization was washed once with a washing solution I (2×SSC, 0.2% SDS) for 10 minutes, and once again with a washing solution II (0.1×SSC) for 10 minutes. After washing, it was air-dried at room temperature or forcibly dried by using a positive displacement air pump for rapid dry, and then kept under shade.

4) Determining HPV Genotypes on the DNA Chip after Washing

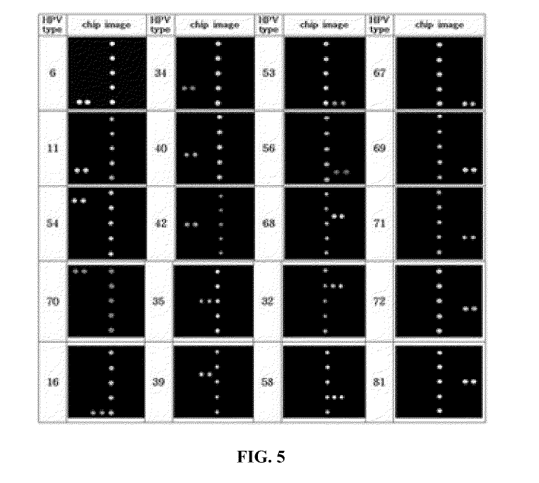

After setting a chip scanner (GSI Lumonics, USA) to operate at 550 nm excitation wavelength and 570 nm emission wavelength, the DNA chip washed in the above 3) was read to identify the location of probes which developed color owing to the binding between the probes for HPV genotype and HPV gene products, determining the HPV genotypes (See, FIGS. 5 and 6).

By providing DNA chips and reagents for a HPV genotype test as a form of a kit, it is possible to easily conduct a HPV genotype test on a multiple amount of samples, while reducing effort and time. A method and a kit for a HPV test are also included in the scope of the present invention to be protected, and a method for providing them in the form of a kit is further illustrated in the following example 3.

### Example 3

### A Kit for Testing HPV Genotype

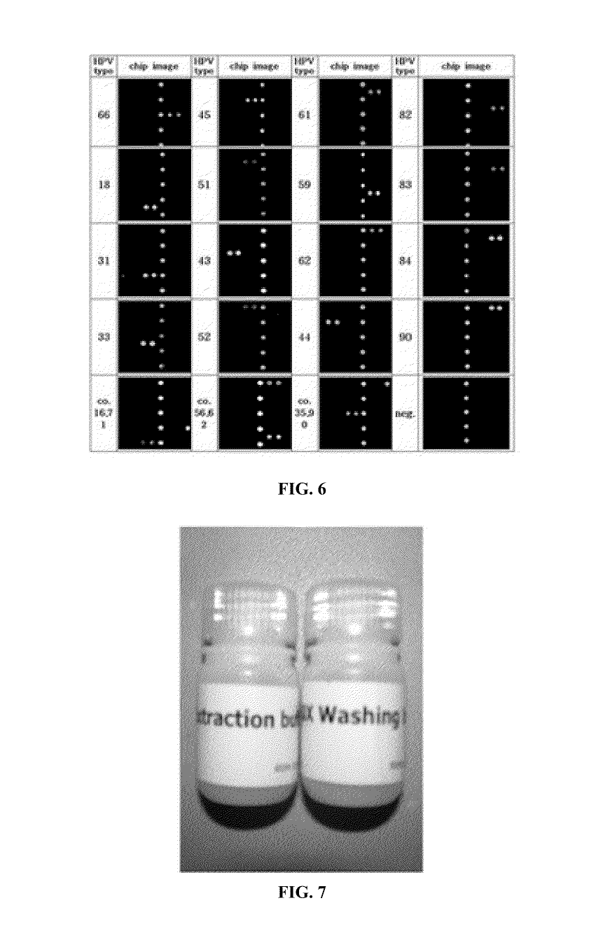

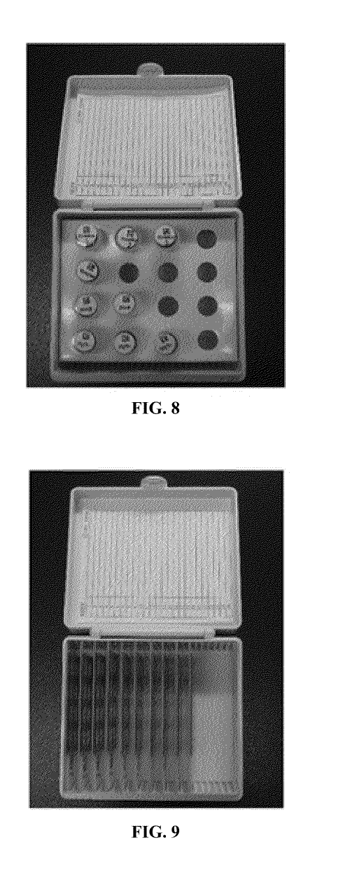

A kit for a HPV genotype test according to the present was composed of 3 parts: a first part including a reagent for DNA extraction from cells taken from the cervix (FIG. 7) a second part including a reagent containing a primer for PCR of HPV genes (FIG. 8); and a third part including a DNA chip for a HPV genotype test which could determine genotype of the PCR products of HPV genes.

3-1:Preparation of a DNA Extraction Kit

With the reagents prepared as it has been described in examples 1 and 2, a kit was formed, wherein the kit was comprised of \textcircled{1} a 30 ml volume tube of 10× washing solution, which contained 20 ml PBS solution, and \textcircled{2} a 30 ml volume tube containing a proteinase K (Sigma, US) at the concentration of 100 μg of the DNA extraction solution/ml, 7% chelex, both of them being kept at room temperature. The kit can process 80 cervical cell samples, and the method for operation and use the same was as described in the examples 1 and 2.

3-2: Preparation of a Kit with Reagents for PCR of HPV DNA Amplification

As described in example 2, reagents for PCR of HPV genes were provided as a kit.

The kit was comprised of: \textcircled{1} 20 pmol/μl of primer selected from SEQ.ID.NO:1 to SEQ.ID.NO:6, these are PCR primers for HPV genes, \textcircled{2}a 10×PCR buffer solution, \textcircled{3} a mixed solution of 2.5 mM dNTP (dATP, dGTP, dCTP, dUTP), \textcircled{4} an 1.5 ml volume tube containing 100 ul of a beta-globin primer at the concentration of 3 pmol/μl, \textcircled{5} a premix of primers for a HPV test, wherein the premix contained uracil-N-glycosylase (1 U/μl) 20 ul and template DNA 5 ul, and as a control, \textcircled{6} a 1.5 ml tube containing Taq. DNA polymerase 250 units (Solgent, South Korea), \textcircled{7} a 1.5 ml tube containing a 2× hybridization solution, \textcircled{8} a 1.5 ml tube containing distilled water 1 ml. The kit containing all the above-described reagents was kept in a freezer at −20° C., and the method of use was the same as described in example 2.

3-3: Preparation of a Kit with a DNA Chip of HPV Genes

With one HPV DNA chip fabricated as in example 2, it was possible to test 8 samples. A kit was composed of 10 pieces of such DNA chips. A hybridization chamber was attached to the kit, for ready use, and the method of use was the same as described in example 2. The DNA chip kit for a HPV genotype test was composed of 10 DNA chips, being capable of processing 80 cervical cell samples (FIG. 9).

### Example 4

### Comparison Between the DNA Chip Method and a Base Sequencing Method

Based on the results obtained from the DNA chip according to the present invention, each HPV genotype was determined. For confirming the genotype results determined by the DNA chip according to the present invention, a base sequence analysis was carried out.

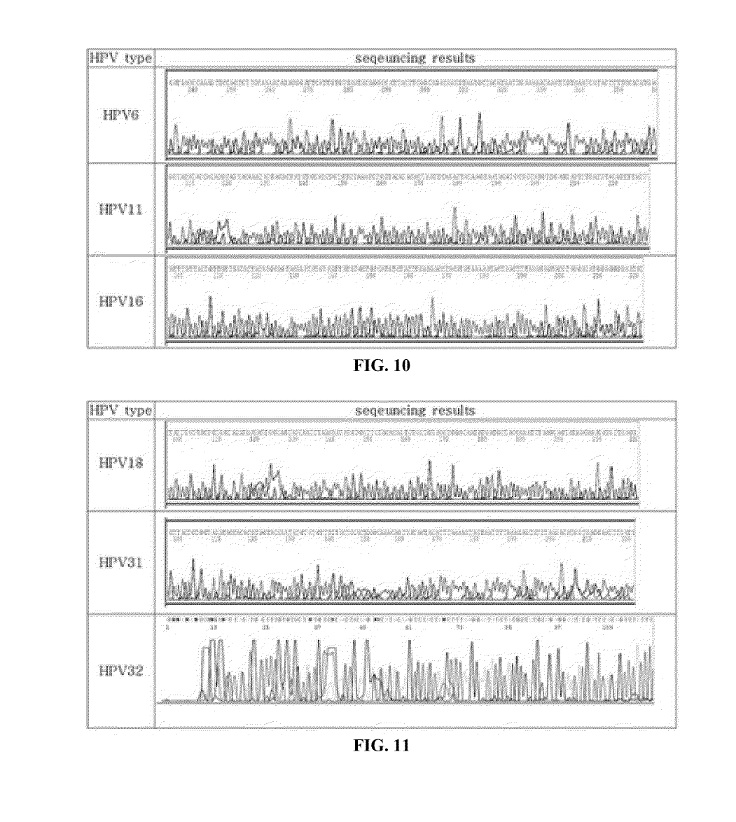

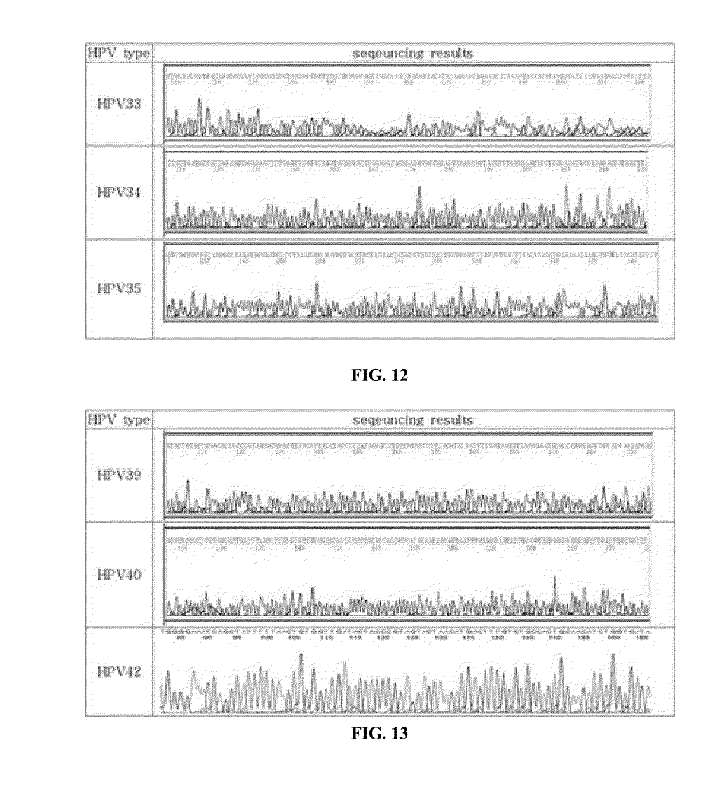

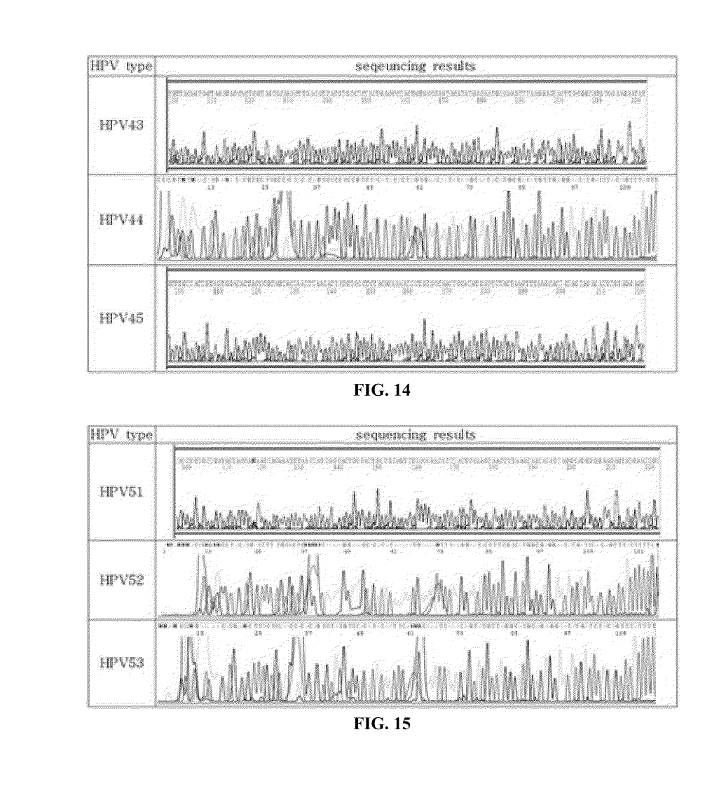

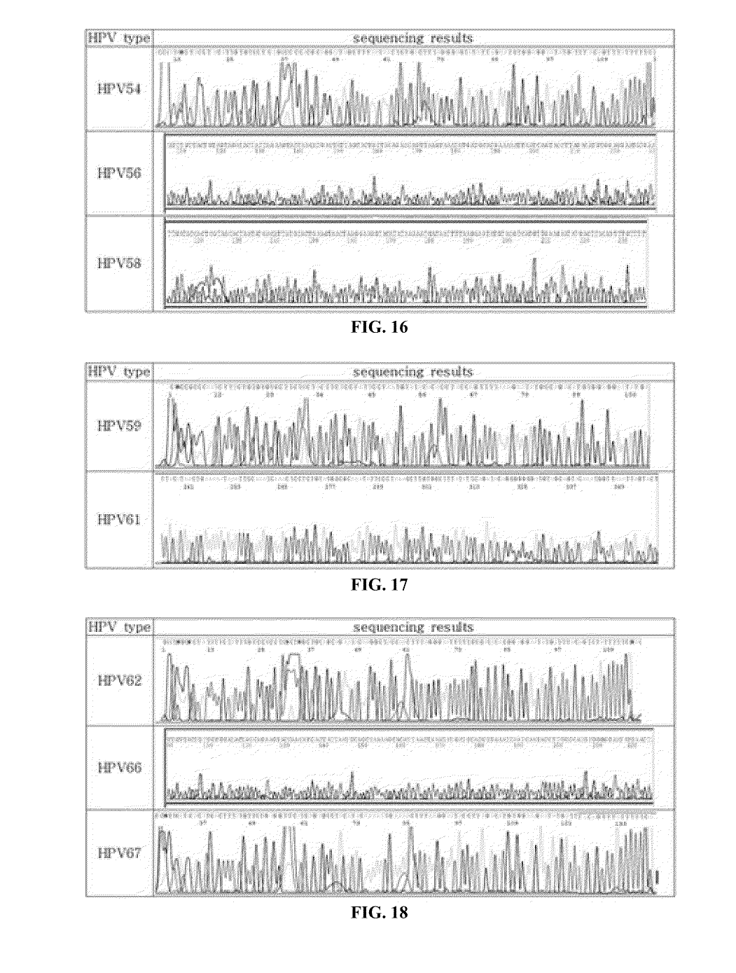

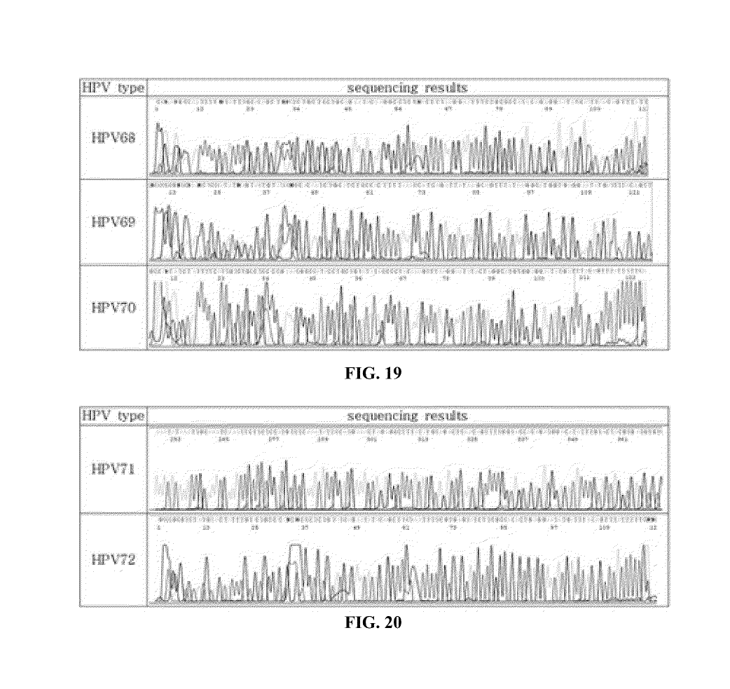

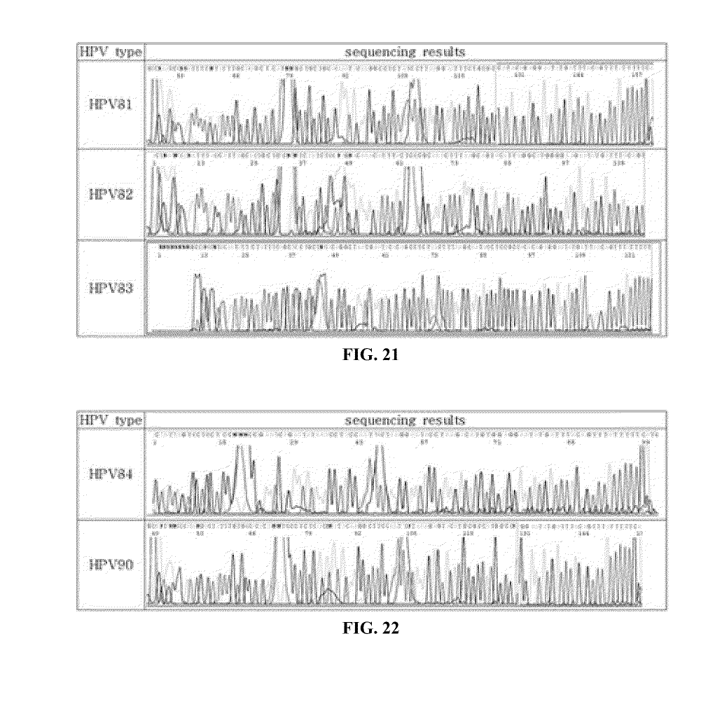

To 5 μl of the PCR product of each sample corresponding to the HPV genotype of the HPV DNA chip (PCR products of Example 1-1), 8 μl of a reagent (Bigdye), 1 μl of 5 primer of each genes and the like were added, making a mixture to have the total volume of 20 μl. The mixture was denatured at 95° C. for 2 minutes. Sequence analysis was conducted at 95° C., 20 seconds, 50° C., 5 seconds and 60° C., 2 minutes, 25 times, and then determined the base sequence (ABI3100, Applied Biosystems). The results were shown in FIGS. 10 to 22. The results obtained from sequence analysis all agreed with the HPV genotype results from the HPV DNA chip method of the present invention.

1. A primer for HPV (Human Papilloma Virus) amplification, comprising at least one base sequence selected from the group consisting of SEQ. ID.NO: 1 to SEQ.ID.N0:6.

2. The primer for HPV amplification according to claim 1, wherein the base sequence has a fluorescent dye attached to 5′ end thereof.

3. The primer for HPV amplification according to claim 2, wherein the fluorescent dye is selected from the group consisting of Cy3, Cy5, a biotin binding material, Alexa 647, Alexa 555, 5-(2-aminoethyl)amino-1-naphthalene sulfonic acid (EDANS), tetramethyl rhodamine (TMR), tetramethyl rhodamine isocyanate (TMRITC), fluorescein isocyanate (FITC), χ-rhodamine and Texas red.

4. A probe which forms complementary binding with HPV, comprising at least one oligonucleotide selected from the group consisting of oligonucleotide having base sequences represented as SEQ.ID.N0:9 to SEQ.ID.N0:44, and oligonucleotide having base sequences complementary to said sequences.

5. The probe which forms complementary binding with HPV according to 4, wherein the oligonucleotide corresponds to the following HPV types: HPV-6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84 and 90.

6. The probe which forms complementary binding with HPV according to 4,wherein an amine group is attached to the 5′ end of the base sequences represented by SEQ.ID.N0:9 to SEQ.ID.N0:44.

7. A DNA chip for testing HPV genotypes, comprising a probe according to claim 4.

8. A kit for detecting or testing HPV genotype, comprising:
a reagent for DNA extraction;
a PCR primer for amplification of the extracted DNA; and
a DNA chip for testing HPV genotype comprising a probe according to claim 4.

9. The kit for detecting or testing HPV genotype according to claim 8, wherein the reagent for DNA extraction comprises proteinase and chelex 7% (w/v).

10. The kit for detecting or testing HPV genotype according to claim 8, wherein the primer for PCR comprises at least one base sequence selected from the group consisting of SEQ.ID.NO:1 to SEQ.ID.N0:6.

11. The kit for detecting or testing HPV genotype according to claim 8, wherein the primer for PCR is one selected from: JK1 set consisting of SEQ. ID.NO: 1 and SEQ.ID.N0:2 JK2 set consisting of SEQ.ID.N0:3 and SEQ.ID.N0:4; and JK3 set consisting of SEQ.ID.N0:5 and SEQ.ID.N0:6.

12. A method for testing HPV genotype, comprising the steps of
extracting DNA from cells taken from the cervix;
amplifying the DNA by PCR involving a primer for HPV amplification;
hybridization of the amplified DNA to a DNA chip comprising a HPV probe which comprises at least one oligonucleotide selected from the group consisting of oligonucleotide having base sequences represented as SEQ.ID.N0:9 to SEQ. ID.NO′•44 and oligonucleotide having base sequences complementary to said sequences; and determining the HPV genotype through the product specifically hybridized with the probe.

13. The method for testing HPV genotype according to 12, further comprising, in the PCR amplification, a beta-globin primer set represented by SEQ.ID.N0:7 and SEQ.ID.N0:8, as a control.

14. The method for testing HPV genotype according to 12, wherein, in the hybridization of the amplified DNA, an amine group is attached to the 5′ end of the base sequences represented by SEQ.ID.N0:9 to SEQ.ID.N0:44.

15. The method for testing HPV genotype according to 12, wherein, in the step of determining the HPV genotype, the HPV genotype is selected from the group consisting of HPV-6, 11, 16, 18, 31, 32, 33, 34, 35, 39, 40, 42, 43, 44, 45, 51, 52, 53, 54, 56, 58, 59, 61, 62, 66, 67, 68, 69, 70, 71, 72, 81, 82, 83, 84, 90 and mixtures thereof.


SEQUENCE LISTING (44 sequences)

SEQ ID NO: 1 - DNA, 20 bp, Artificial Sequence
  Primer for cloning HPV gene
agtggstctatggtwwcytc

SEQ ID NO: 2 - DNA, 21 bp, Artificial Sequence
  Primer for cloning HPV gene
catagtatgtawrtakgycat

SEQ ID NO: 3 - DNA, 21 bp, Artificial Sequence
  Primer for cloning HPV gene
gccacaayaatggyatwtgyt

SEQ ID NO: 4 - DNA, 23 bp, Artificial Sequence
  Primer for cloning HPV gene
tgtaaatcatattcctcmmcatg

SEQ ID NO: 5 - DNA, 21 bp, Artificial Sequence
  Primer for cloning HPV gene
ccacctataggggaacactgg

SEQ ID NO: 6 - DNA, 20 bp, Artificial Sequence
  Primer for cloning HPV gene
gaggtaaccatagaaccact

SEQ ID NO: 7 - DNA, 22 bp, Artificial Sequence
  Beta globin primer
caagacaggtttaaggagacca

SEQ ID NO: 8 - DNA, 22 bp, Artificial Sequence
  Beta globin primer
ggttggccaatctactcccagg

SEQ ID NO: 9 - DNA, 30 bp, Artificial Sequence
  Probe for HPV6
accaattctgattataaagagtacatgcgt

SEQ ID NO: 10 - DNA, 30 bp, Artificial Sequence
  Probe for HPV11
ttcagattataaggaatacatgcgccatgt

SEQ ID NO: 11 - DNA, 30 bp, Artificial Sequence
  Probe for HPV16
ctacatataaaaatactaactttaaggagt

SEQ ID NO: 12 - DNA, 30 bp, Artificial Sequence
  Probe for HPV18
ctcctgtacctgggcaatatgatgctacca

SEQ ID NO: 13 - DNA, 30 bp, Artificial Sequence
  Probe for HPV31
ctacatttaaaagtagtaattttaaagagt

SEQ ID NO: 14 - DNA, 30 bp, Artificial Sequence
  Probe for HPV32
gtgctactgtaacaactgaagacacataca

SEQ ID NO: 15 - DNA, 30 bp, Artificial Sequence
  Probe for HPV33
cacacaagtaactagtgacagtacatataa

SEQ ID NO: 16 - DNA, 30 bp, Artificial Sequence
  Probe for HPV34
tgcaaacagtaattttaaggaatacctcag

SEQ ID NO: 17 - DNA, 30 bp, Artificial Sequence
  Probe for HPV35
ctgctgtgtcttctagtgacagtacatata

SEQ ID NO: 18 - DNA, 30 bp, Artificial Sequence
  Probe for HPV39
tgatccttctaagtttaaggaatataccag

SEQ ID NO: 19 - DNA, 30 bp, Artificial Sequence
  Probe for HPV40
taataacagtaatttcaaggaatatttgcg

SEQ ID NO: 20 - DNA, 30 bp, Artificial Sequence
  Probe for HPV42
cagctgctaattttaaggaatatttaagac

SEQ ID NO: 21 - DNA, 30 bp, Artificial Sequence
  Probe for HPV43
tatgacaatgcaaagtttaaggaatacttg

SEQ ID NO: 22 - DNA, 30 bp, Artificial Sequence
  Probe for HPV44
tactagtgaacaatataagcaatacatgcg

SEQ ID NO: 23 - DNA, 30 bp, Artificial Sequence
  Probe for HPV45
cctactaagtttaagcagtatagtagacat

SEQ ID NO: 24 - DNA, 30 bp, Artificial Sequence
  Probe for HPV51
tccaagtaactttaagcaatatattaggca

SEQ ID NO: 25 - DNA, 30 bp, Artificial Sequence
  Probe for HPV52
tgaaaattttaaggaataccttcgtcatgg

SEQ ID NO: 26 - DNA, 30 bp, Artificial Sequence
  Probe for HPV53
agcaaattaaacagtatgttagacatgcag

SEQ ID NO: 27 - DNA, 30 bp, Artificial Sequence
  Probe for HPV54
ttctgactttagggagtatattagacatgt

SEQ ID NO: 28 - DNA, 30 bp, Artificial Sequence
  Probe for HPV56
tgcacgaaaaattaatcagtaccttagaca

SEQ ID NO: 29 - DNA, 30 bp, Artificial Sequence
  Probe for HPV58
aaaaatgataattttaaggaatatgtacgt

SEQ ID NO: 30 - DNA, 30 bp, Artificial Sequence
  Probe for HPV59
cacacctaccagttttaaagaatatgccag

SEQ ID NO: 31 - DNA, 30 bp, Artificial Sequence
  Probe for HPV61
tgtatctgaatataaagccacaagctttag

SEQ ID NO: 32 - DNA, 30 bp, Artificial Sequence
  Probe for HPV62
tccactgctgcagcagaatacaaggctacc

SEQ ID NO: 33 - DNA, 30 bp, Artificial Sequence
  Probe for HPV66
atgatgcccgtgaaatcaatcaataccttc

SEQ ID NO: 34 - DNA, 30 bp, Artificial Sequence
  Probe for HPV67
tgttctgaggaaaaatcagaggctacatac

SEQ ID NO: 35 - DNA, 30 bp, Artificial Sequence
  Probe for HPV68
ttatgatcctaataaatttaaggaatatat

SEQ ID NO: 36 - DNA, 30 bp, Artificial Sequence
  Probe for HPV69
taaaccatcagattataagcagtttataag

SEQ ID NO: 37 - DNA, 30 bp, Artificial Sequence
  Probe for HPV70
tagccctacaaagtttaaggaatatactag

SEQ ID NO: 38 - DNA, 30 bp, Artificial Sequence
  Probe for HPV71
gcctctagtttcatggaatatttgagacat

SEQ ID NO: 39 - DNA, 30 bp, Artificial Sequence
  Probe for HPV72
gcgtcctctgtatcagaatatacagcttct

SEQ ID NO: 40 - DNA, 30 bp, Artificial Sequence
  Probe for HPV81
ggcctctaactttaaggaatttctgcgcca

SEQ ID NO: 41 - DNA, 29 bp, Artificial Sequence
  Probe for HPV82
ccatctgttgcacaaacatttactccagc

SEQ ID NO: 42 - DNA, 30 bp, Artificial Sequence
  Probe for HPV83
cacaggctaatgaatacacagcctctaact

SEQ ID NO: 43 - DNA, 30 bp, Artificial Sequence
  Probe for HPV84
caccgaatcagaatataaacctaccaattt

SEQ ID NO: 44 - DNA, 30 bp, Artificial Sequence
  Probe for HPV90
gacacatacaaggcttccaattttaaagag